(19) United States
(12) Patent Application Publication
(10) Pub. No.: US 2002/0004525 A1
(43) Pub. Date: Jan. 10, 2002

(54) Treatment of multiple sclerosis

(76) Inventor: Jack Colover, London (GB)

Correspondence Address: STEVENS, DAVIS, MILLER & MOSHER, L.L.P., Suite 850, 1615 L Street, N.W., Washington, DC, 20036, US

(21) Appl. No.: 09/877,118

(22) Filed: Jun. 11, 2001

(30) Foreign Application Priority Data
Jun. 12, 2000 (GB) 00/14356

(51) Int. Cl. A61K 31/5585; A61K 31/557

(52) U.S. Cl. 514/461; 514/573

(57) ABSTRACT

Multiple sclerosis is treated by reducing the fluid pressure in the myelin sheath of nerves to reduce or prevent bursting of the sheath. Prostaglandins are the preferred agent for achieving a reduction in the pressure.

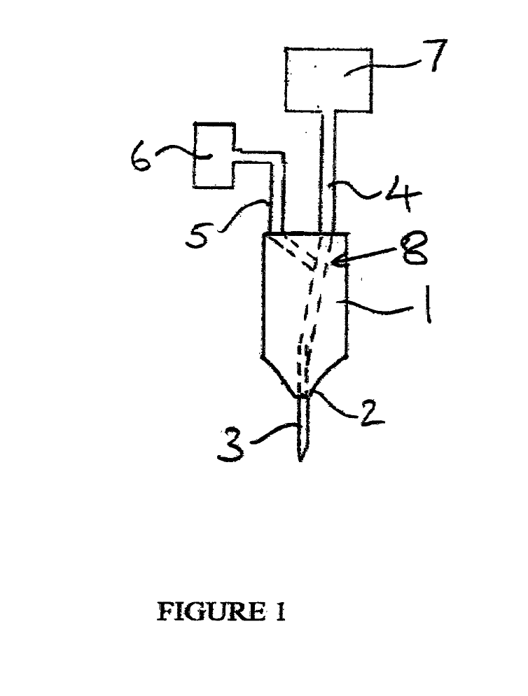

This invention relates to the treatment of demyelinating diseases, for example multiple sclerosis, and to pharmaceutical compositions useful therefor.

Multiple sclerosis is a chronic incurable disease of human beings for which presently known treatments have no effect or only marginal therapeutic responses. It is a demyelinating disease, i.e. the myelin sheaths surrounding the nerve fibres are destroyed, leading to severe disability, paralysis, mental illness and visual disturbances. It may be chronically progressive or relapsing in which case each relapse is associated with marked deterioration. Much of the myelin loss is due to auto-immune reactions, which destroy the myelin sheaths of the nerve fibres in the central nervous system. There are certain other generally similar demyelinating diseases.

I have made some study of this matter and have found that very surprisingly, the cylindrical myelin sheath around nerve axons is maintained in its normal shape partly by fluid under pressure therein. That is to say, the sheath is “inflated” by hydrostatic or hydrodynamic pressure, and it is the maintenance of an appropriate pressure within the sheath that assists in keeping it in its normal shape.

I have filter noted that, in patients suffering from multiple sclerosis, at times of relapse when the symptoms are increased, it is as if the “inflated” sheath had swollen and burst, thus releasing proteins and other material therefrom. In such circumstances, I believe that the burst may occur as a result of a malfunction of the body's control system for maintaining a normal pressure within the sheath. Nothing is yet known of any such pressure control system. The escape of sheath components leads to auto-immune reactions which destroy the myelin. These auto-immune reactions do not occur so long as the myelin proteins are contained within the sheath, but when they escape in this illness the immune system reacts very vigorously against them. I have found that it is release of the proteins upon bursting of the sheath which provokes relapses.

In its broadest aspect, my invention resides in treating multiple sclerosis and other demyclination diseases of the central nervous system, by control of the intra-sheath fluid pressure to bring it below sheath-bunting point. In this way, the risk of bursting due to elevated pressure within the sheath is eliminated, or very substantially reduced.

There are a number of types of drugs which are known to reduce body fluid pressures in various circumstances. One of the most promising is the prostaglandins used to assist in the control of the intraocular fluid pressure associated with glaucoma including, for example, brimonidine, latanoprost, PGE1, PGE2 or PGE3.

Accordingly, in a farther aspect, the invention provides a method of treating multiple sclerosis and other demyclination diseases of the nervous system, which comprises using an agent to, or administering a prostaglandin or other drug to, control the intra-sheath fluid pressure.

The invention also includes the use of an agent, prostaglandin or other drug to control the internal myelin sheath fluid pressure.

The invention further includes the use of a prostaglandin or other drug to make a pharmaceutical composition for the control of the internal myelin sheath fluid pressure.

The pharmaceutical compositions of the invention can be in any appropriate form, e.g. for administration orally or by injection. Oral formulations include tablets, capsules and liquids for example. Preferably, administration is by injection intramuscularly. The adult dose of prostaglandin (or other drug) will normally be in tie range from about 100 to 500 mg, preferably about 250 mg, which will be effective for up to about 12 hours. Regular administration e.g. at least once and possibly more often per day will be needed.

The concept of monitoring and/or measuring the intra-sheath fluid pressure as a diagnostic technique is new and forms a further aspect of the invention. There are various ways in which the intra-sheath fluid pressure can be measured. In one preferred procedure which I have developed, a probe is inserted into the myelin sheath or nerve to sense the fluid pressure therein.

I prefer to use a hollow hypodermic needle of small bore which includes a pressure sensor in fluid communication with the bore of the needle. The needle filled with fluid (e.g. saline) is inserted into the nerve trunk and the fluid pressure therein is communicated to the pressure sensor.

In a further aspect, the invention includes a device for sensing the fluid pressure in the myelin sheath or nerve, which device comprises a probe such as a hypodermic needle for insertion into the sheath or nerve, and a pressure sensor in fluid communication with the probe, e.g. the bore of said needle, to sense the fluid pressure in the myelin sheath or nerve adjacent to the needle.

In order that the invention may be more fully understood, reference is made to the accompanying drawing which is a schematic view of one example of hypodermic needle device for use in measuring intra-sheath fluid pressure.

Referring to the drawing, the device comprises a body 1, at one end 2 of which is mounted a hypodermic needle 3 with a bevelled tip. The bore of the needle is connected to a fluid supply line 4 to supply fluid into the needle from a pump or reservoir 7. Within body 1, a fluid pressure sensor line 5 connects into supply line 4 at junction 8 (see phantom lines). Sensor line 5 communicates with a fluid pressure sensor 6.

In use, saline or other suitable fluid is pumped through line 4 to fill needle 3, and to back-fill (as necessary) line 5 to ensure fluid communication between needle 3 and sensor 6. The needle is then inserted into the myelin sheath or nerve. The intra-sheath fluid pressure is communicated through the fluid in needle 3 and line 5 back to the sensor 6.

In vitro experimental work to date indicates in the peripheral nerve a normal myelin intra-sheath fluid pressure in a pig is of about 30 mm Hg. The normal fluid pressure in a human myelin sheath is believed to be not very different. Pressure measurements have been made, using an optic-fibre tipped pressure transducer, on the sciatic nerves of pigs starting immediately after they were killed by barbiturate injection, whilst anaesthetised by oxygen, nitrous oxide and halothane. The pressure recorded at onset was 30 mm Hg and it fell to zero in 30 minutes. During this time, the pressure in the nearby gluteal muscles was zero.

Open perfusion micro-incubators can be used for tests on nerve tissue to measure in vitro the intra-sheath fluid pressure. Also, the tissue can be perfused with prostaglandin and the pressure reduction monitored. One suitable micro-incubator is a Leiden micro-incubator available from Harvard Apparatus Ltd. The pressure is measured by using a small hypodermic needle which is filled with saline and which includes a pressure sensor. The needle is then introduced into the nerve trunk to monitor the pressure therein.

Tests on rabbit nerve tissue have shown that the fluid pressure inside the perineurium (which is the same as the intra-sheath pressure) is very similar to that measured in pigs ire. 30 mm Hg. I believe the normal (healthy) intra-sheath pressure in human nerve tissue will be about the same.

The therapy which I propose according to my invention to prevent the harmful relapses in multiple sclerosis is novel, that is, to lower the pressure inside the myelin sheaths so that the bursting does not occur. This method of treating the disease so as to arrest it and halt the usual deterioration has never been used or suggested previously. This very simple explanation of the pathological processes involved is the key to this therapy. In the vast majority of cases, one can expect severe deterioration over the years, the patients eventually dying from renal failure or infection, lung infections or severe bed sores.

1. A method of treating multiple sclerosis and other demyclination diseases of the nervous system, which comprises using an agent to, or administering a prostaglandin or other drug to, control the intra-sheath fluid pressure to reduce or avoid bursting of the myelin sheath around nerves.

2. A method according to claim 1, which comprises administering a prostaglandin to reduce the intra-sheath fluid pressure.

3. A method according to claim 2, wherein the prostaglandin is latanoprost, brimonidine, PGE1, PGE2 or PGE3.

4. A pharmaceutical composition for the treatment of multiple sclerosis and other demyclination diseases of the nervous system, which comprises a pharmaceutical substance effective to reduce the fluid pressure within the myelin sheath of nerves, and a pharmaceutically acceptable carrier therefor.

5. A composition according to claim 4, wherein the substance is a prostaglandin.

6. A composition according to claim 5, wherein the prostaglandin is latanoprost, brimonidine, PGE1, PGE2 or PGE3.

7. A composition according to claim 4, 5 or 6, which is for injection administration.

8. The use of a pharmaceutical substance effective to reduce the fluid pressure within the myelin sheath of nerves, to manufacture a composition for the treatment of multiple sclerosis.

9. The use according to clam 8, wherein the substance is a prostaglandin.

10. The use according to claim 9, wherein the prostaglandin is latanoprost, brimonidine, PGE1, PGE2 or PGE3.

11. A composition according to any of claims 4 to 7, wherein the pharmaceutical substance is selected for inclusion in the composition by in vitro testing by perfusion into nerve trunks or fibre, and measurement of the change in intra-sheath fluid pressure therein.

12. The use according to any of claims 8 to 10, wherein the pharmaceutical substance is selected for inclusion in the composition by in vitro testing by perfusion into nerve trunks or fibre, and measurement of the change in intra-sheath fluid pressure therein.

13. A method of testing for the onset of multiple sclerosis, or for the onset of a relapse, which comprises monitoring the intra-sheath fluid pressure in nerve tissue.

14. A method of monitoring the fluid pressure within the myelin sheath of a nerve which comprises introducing into the sheath or nerve trunk a probe and sensing tee fluid pressure thereby.

15. A method according to claim 14, wherein the probe is a hypodermic needle and the fluid pressure is sensed using a fluid pressure sensor in communication with the needle.

16. A device for sensing the fluid pressure in the myelin sheath of a nerve, which device comprises a probe for insertion into the sheath or nerve trunk, and a pressure sensor in communication with the probe to sense the fluid pressure in the myelin sheath of the nerve trunk.

17. A device according to claim 16, wherein the probe is a hypodermic needle.

18. A method of testing whether a pharmaceutical substance is suitable for the treatment of multiple sclerosis, which comprises testing whether it reduces or controls the intra-sheath fluid pressure around nerves.
